(19) United States
(12) Patent Application Publication
(10) Pub. No.: US 2018/0214571 A1
(43) Pub. Date: Aug. 2, 2018

(54) LIPOSOME LOADED WITH MAGNETIC MICROPARTICLES FOR TARGETED DELIVERY OF STEM CELLS

(71) Applicants: The Board of Trustees of the Leland Stanford Junior University, Stanford, CA (US); The Regents of the University of California, Oakland, CA (US)

(72) Inventors: Jayakumar Rajadas, Cupertino, CA (US); Mohammadreza Mohammadi, Irvine, CA (US); Mohammed Inayathullah, Santa Clara, CA (US); Geoffrey C. Gurtner, Woodside, CA (US); Tejal Ashwin Desai, San Francisco, CA (US)

(21) Appl. No.: 15/884,736

(22) Filed: Jan. 31, 2018

Related U.S. Application Data
(60) Provisional application No. 62/453,423, filed on Feb. 1, 2017.

(51) Int. Cl. A61K 47/69; A61K 35/12; A61K 47/52; A61N 2/00

(52) U.S. Cl. CPC A61K 47/6911; A61N 2/002; A61K 47/52; A61K 35/12

(57) ABSTRACT

Compositions and methods are provided for delivery of stem cells to a targeted tissue. A population of stem cells, including for example a regenerative stem cell population, is bound to magnetic vesicular particles. The magnetic vesicular particles comprise one or more magnetic nanoparticles within a lipid membrane, e.g. a liposomal structure. The cells are delivered to a targeted tissue by application of a magnetic field.

### CROSS REFERENCE

This application claims benefit of U.S. Provisional Patent Application No. 62/453,423, filed Feb. 1, 2017, which application is incorporated herein by reference in its entirety.

### BACKGROUND OF THE INVENTION

Regenerative medicine is the process of creating living, functional tissues to repair or replace tissue or organ function lost due to age, disease, damage, or congenital defects. This field holds the promise of regenerating damaged tissues and organs in the body by stimulating previously irreparable organs to heal themselves. Regenerative medicine also empowers scientists to grow tissues and organs in the laboratory and safely implant them when the body cannot heal itself. Importantly, regenerative medicine has the potential to solve the problem of the shortage of organs available through donation compared to the number of patients that require life-saving organ transplantation.

An important feature of regenerative medicine involves the use of stem cells. Stem cells have a capacity both for self-renewal and the generation of differentiated cell types, which provides the possibility for therapeutic regeneration of cells and tissues in the body. There are many types of stem cells. Each type plays a different role in the body as we grow and develop. Some stem cells only exist for a limited period, such as during the development of an embryo. Others are only found in specific parts of the body, such as in hair follicles or the liver. Depending on the purpose and location of the stem cells, there may be limitations to what cell types the stem cell can differentiate into. Regenerative stem cell populations include somatic stem cells that resident in adult tissues; pluripotent stem cells generated from embryonic tissues or induced by the introduction of specific reprogramming factors, and tissue specific stem cells derived therefrom.

Somatic, or adult, stem cells are undifferentiated cells that reside in differentiated tissues, and have the properties of both self-renewal and generation of differentiated cell types. The differentiated cell types may include all or some of the specialized cells in the tissue. For example, hematopoietic stem cells give rise to all hematopoietic lineages, but not stromal and other cells found in the bone marrow. Sources of somatic stem cells include bone marrow, blood, the cornea and the retina of the eye, brain, skeletal muscle, cartilage, bones, dental pulp, liver, skin, the lining of the gastrointestinal tract, and pancreas, and the like. Adult stem cells are usually quite sparse. Often they are difficult to identify, isolate, and purify. Often, somatic stem cells are quiescent until stimulated by the appropriate growth signals.

Currently bone marrow transplants (also called hematopoietic stem cell transplants) are in clinical use for treating blood and disorders in the immune system. Other stem cell treatments include emergency skin grafts using skin (epidermal) stem cells, and repair of the cornea of the eye using limbal stem cells. However, many stem cell treatments are being researched and several show promise in clinical trials. For example, adipose tissue-derived SCs transplantation has attracted great consideration as a therapeutic tool to treat various diseases such as cardiovascular diseases, liver and renal diseases, and neurological diseases (see Liao et al. (2016) Sci. Rep. 6:18746).

Methods have been developed for stem cell delivery; however, these currently lack certain vital characteristics, and are not ideal. Traditional injection methods for cell delivery, which are popular with animal models, often result in poor cell survival and low levels of cell integration into the host tissue, see Duscher et al. (2016) Gerontology 62:216-225. Another major difficulty in stem cell delivery based therapies is due to their tendency to get delocalized from an injury site over time (Cores et al. (2015) J. Funct. Biomater. 6:526-546). Active research in this area includes incorporating biomaterials, novel culturing strategies, and surgical devices into delivery methods to help cells survive and integrate appropriately into the human body, as well as maintaining the SCs localization for the duration of the therapy.

For clinical translation, stem cell delivery presents fundamental challenges due to a restriction in tissue targeting, and a high attrition rate with systemic targeting. Local delivery has been a focus of research on SC delivery (see Falanga et al (2007) Tissue Eng 13:1299-1312). However, oxidative stress hypoxia, and inflammation within the wound can provide an extremely hostile environment for delivered cells. In addition, introduction of shear injury during injection may impede the cell engraftment. Cell engraftment following wound injection has been reported to be as low as 0% at 11 days in preclinical models, potentially from shear injury during injection (see Garg et al. (2014) Stem Cells Transl Med. 3(9): 1079-1089).

Some methods have been suggested to overcome such imperfections. For instance, to deliver the cells into the injured part of myocardium without open chest surgery, Cheng et al. (2015) Nature Communications, vol. 5, article 4880, modified SPIONs, labeled cells, and injected them to random parts of the preinfarcted area. Specifically, they doubly conjugated an FDA-approved SPION (i.e., ferumoxytol, an intravenous iron product replacement used to treat anemia) with anti-CD45 (specific to exogenous bone marrow-derived stem cells) and with antibodies found in injured cardiomyocytes (myosin light chain). The dual antibody conjugated nanoparticles enabled high affinity binding of therapeutic cells to injured cardiomyocytes both in vitro and in vivo. The obtained results report that this approach can target acute myocardial infarction. However, a major impediment of the SPION-labeling strategy is the leakage of SPION into adjacent cells, mostly through exocytosis (Sakhtianchi et al. (2013) Advances in Colloid and Interface Science, vol. 201-202, pp. 18-29) and advanced dilution after mitosis and poor localization in the myocardial interstitial tissue Amsalem et al. (2007) Circulation, vol. 116, no. 11, pp. I-38-I-45; Terrovitis et al. (2008) Circulation, vol. 117, no. 12, pp. 1555-1562, 2008. Another main drawback associated with current SPION technology is its inability to distinguish between viable and nonviable cells (Santoso and Yang (2016) Stem Cells International, pp. 1-9).

Moreover, recent studies have focused on introducing nanoparticles into stem cells through endocytosis. Since the amount of nanoparticles entering into the SCs successfully is limited, the signal strength generated by cells is not sufficient enough for proper tracking or imaging. The SPIONs may also be cytotoxic when endocytosed, which toxicity is proportional to their concentration inside the cells. For instance, myocardial injections consistently carry the risk of vascular embolism.

Therefore, the ability to deliver stem cells to a target tissue, enhance stem cell signaling, and limit delocalization are key aspects in improving stem cell related products. The present invention addresses these issues.

### SUMMARY

Compositions and methods are provided for delivery of stem cells to a targeted tissue. In the methods of the invention, a population of stem cells, including for example a regenerative stem cell population, is bound to magnetic nanovesicles. The magnetic vesicles comprise one or more magnetic nanoparticles within a lipid membrane, e.g. a liposomal structure. The magnetic nanoparticles are nano-level size nanoparticles of ferrite (solid solution of Fe₃O₄ and γ-Fe₂O₃). The liposomes comprising magnetic nanoparticles are bound to the stem cells through covalent or non-covalent binding.

In some embodiments of the invention, magnetic vesicles are covalently bound to the surface of a stem cell. In some embodiments, the vesicle is linked via a primary amine present on the vesicle to a carboxylic acid present on the stem cell surface. In some such embodiments, the methods utilize the water-soluble carbodiimide 1-ethyl-3-(3-dimethylaminopropyl)carbodiimide (EDC) as a carboxyl activating agent for the coupling of primary amines to yield amide bonds. In other embodiments the vesicle is linked by a thioether (e.g. maleimide plus sulfhydryl) to the stem cell surface. In other embodiments an avidin/streptavidin system is used bind the magnetic vesicles to the stem cell.

Stem cells bound to magnetic vesicles of the invention are suspended in a pharmaceutically acceptable excipient, and can be introduced into a subject by local or systemic delivery. The cells are localized at a targeted site by application of a magnetic force at the targeted site.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1D. Magnetic nanoparticles-loaded liposomes for stem cell delivery.

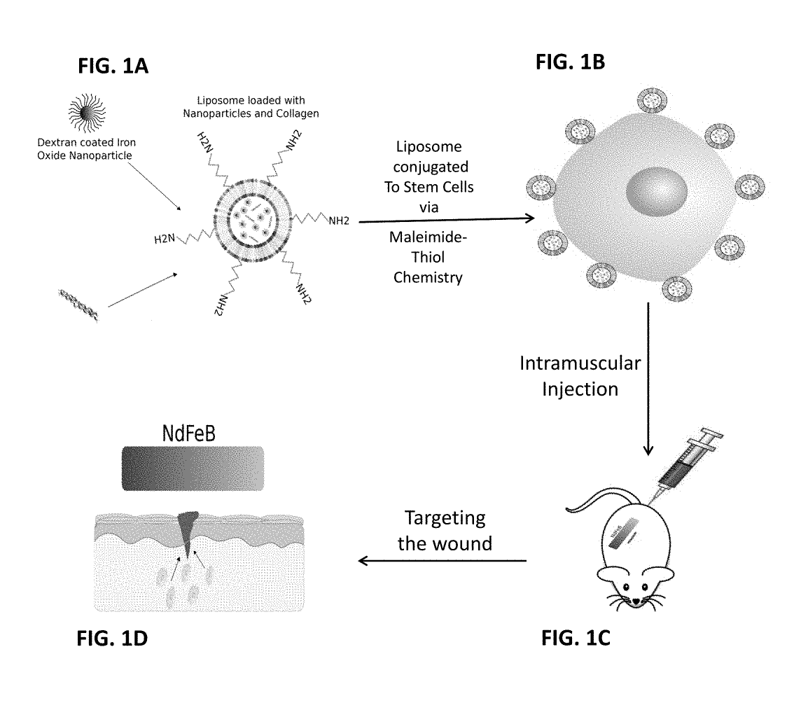

FIG. 2. Schematic of Maleimide-Thiol chemistry used to conjugate liposomes to stem cells.

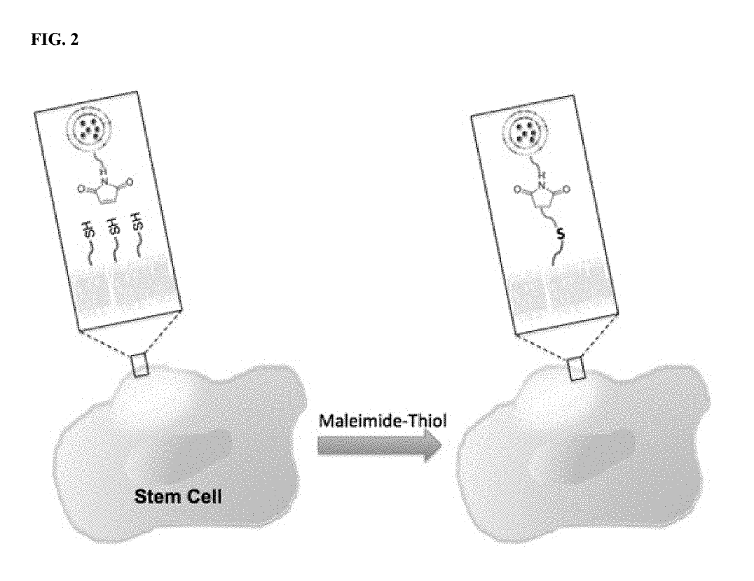

FIG. 3. Applications of magnetic liposomes to deliver stem cells to desired targets through magnetically-directed technology.

FIG. 4A-4B. Transmission Electron Microscopy FIG. 4A and Scanning Electron Microscopy FIG. 4B of magnetic nanoparticles.

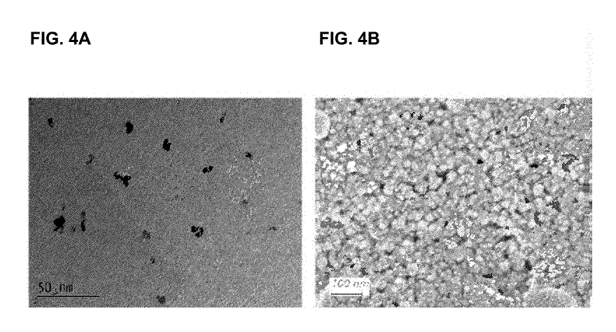

FIG. 5A-5D. Droplet of liposome before applying the magnet FIG. 5A, and the correspondent optical microscopy image FIG. 5B Droplet of liposome 24 h after applying the magnet FIG. 5C, and the correspondent optical microscopy image FIG. 5D (Scale bar=50 μm).

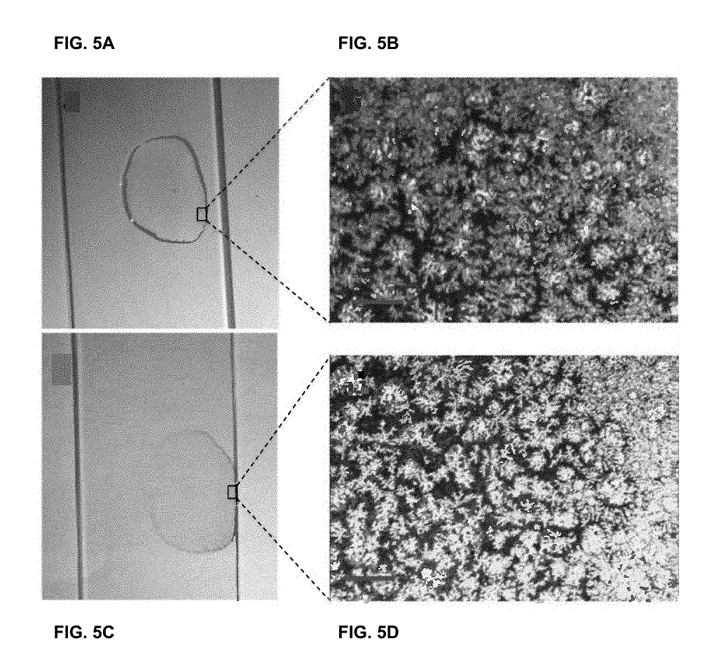

FIG. 6. In vitro guiding of stem cells via magnetic liposomes. Human Embryonic Kidney cells-2913 (HEK-293) were incubated with magnetic liposomes. Magnets were then placed in different positions in vicinity of culture wells.

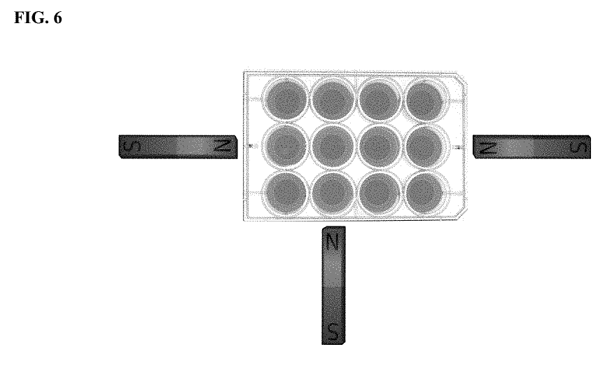

FIG. 7A-7D. In vitro guiding of stem cells via magnetic liposomes. Human Embryonic Kidney cell-2913 (HEK-293) were conjugated with magnetic liposomes. Magnets were then placed in different positions in vicinity of culture wells, and figures show the movement of HEK-293 cells towards magnet. FIG. 7A Control cells without magnetic liposomes. Magnetic liposomes incorporated with stem cells and magnet was placed on the FIG. 7B bottom FIG. 7C left, and FIG. 7D right side of the wells.

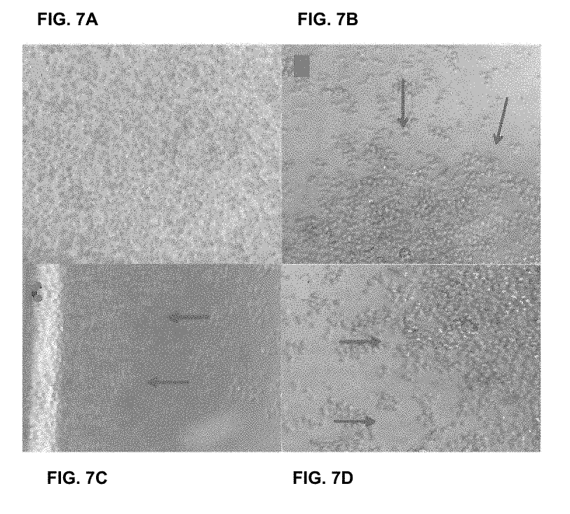

### DETAILED DESCRIPTION

A number of stem/progenitor cells are known in the art, and benefit from the transplantation methods of the invention. These cells include satellite cells in skeletal muscle; hematopoietic stem cells; mesenchymal stem cells; neural stem cells; melanocytes, epidermal stem cells, intestinal stem cells, cardiomyocytes, and the like.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “an”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Compositions and methods are provided for transplantation of stem cells, including pluripotent stem cells, e.g. iPS cells, embryonic stem cells, etc. and for the transplantation of differentiated cells derived from such stem cells, usually derived from such stem cells in vitro.

A cell transplant, as used herein, is the transplantation of one or more cells into a recipient body, usually for the purpose of augmenting function of an organ or tissue in the recipient. As used herein, a recipient is an individual to whom tissue or cells from another individual (donor), commonly of the same species, has been transferred. Generally the MHC antigens, which may be Class I or Class II, will be matched, although one or more of the MHC antigens may be different in the donor as compared to the recipient. The graft recipient and donor are generally mammals, preferably human. Laboratory animals, such as rodents, e.g. mice, rats, etc. are of interest for drug screening, elucidation of developmental pathways, etc. For the purposes of the invention, the cells may be allogeneic, autologous, or xenogeneic with respect to the recipient.

Stem Cell:

The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or asymmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive

Pluripotent stem cells are cells derived from any kind of tissue (usually embryonic tissue such as fetal or pre-fetal tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

Stem cells of interest also include embryonic cells of various types, exemplified by human iPS and human embryonic stem (hES) cells, described by Thomson et al. (1998) Science 282:1145; embryonic stem cells from other primates, such as Rhesus stem cells (Thomson et al. (1995) Proc. Natl. Acad. Sci USA 92:7844); marmoset stem cells (Thomson et al. (1996) Biol. Reprod. 55:254); and human embryonic germ (hEG) cells (Shamblott et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection.

Progenitor or Differentiated Cells.

A “differentiated cell” is a cell that has progressed further down the developmental pathway than the cell it is being compared with. Thus, embryonic stem cells can differentiate to lineage-restricted progenitor cells (such as a mesodermal stem cell), which in turn can differentiate into other types of progenitor cells further down the pathway (such as an cardiomyocyte progenitor), and then to an end-stage differentiated cell, which plays a characteristic role in a certain tissue type, and may or may not retain the capacity to proliferate further. For the purposes of the present invention, progenitor cells are those cells that are committed to a lineage of interest, but have not yet differentiated into a mature cell.

The potential of ES cells to give rise to all differentiated cells provides a means of giving rose to any mammalian cell type, and so a very wide range of culture conditions may be used to induce differentiation, and a wide range of markers may be used for selection. One of skill in the art will be able to select markers appropriate for the desired cell type.

Stem cells may be characterized by both the presence of markers associated with specific epitopes identified by antibodies and the absence of certain markers as identified by the lack of binding of specific antibodies. Stem cells may also be identified by functional assays both and in vivo, particularly assays relating to the ability of stem cells to give rise to multiple differentiated progeny.

Somatic Stem Cells:

Somatic stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells (including without limitation satellite cells as described herein); hematopoietic stem cells and progenitor cells derived therefrom (U.S. Pat. No. 5,061,620); neural stem cells (see Morrison et al. (1999) Cell 96:737-749); embryonic stem cells; mesenchymal stem cells; mesodermal stem cells; liver stem cells, etc.; and the like.

The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

Hematopoietic stem cells (HSCs) have the ability to renew themselves and to give rise to all lineages of the blood. Conditions of the aged that benefit from activation of HSC include, for example, conditions of blood loss, such as surgery, injury, and the like, where there is a need to increase the number of circulating hematopoietic cells. Anemia is an abnormal reduction in red blood cells, which can occur from a malfunction in the production of red blood cells. Weakness and fatigue are the most common symptoms of even mild anemia. Anemia in the elderly is often due to causes other than diet, particularly gastrointestinal bleeding or blood loss during surgery. Anemia in older people is also often due to chronic diseases and folic acid and other vitamin deficiencies.

Neural stem cells are primarily found in the hippocampus, and may give rise to neurons involved in cognitive function, memory, and the like. Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny.

Stem cells may also be present in the epidermis, giving rise both to epidermal and mesenchymal tissues. Like all the body's tissues, the skin undergoes many changes in the course of the normal aging process. The cells divide more slowly, and the inner layer of the dermis starts to thin. Fat cells beneath the dermis begin to atrophy. In addition, the ability of the skin to repair itself diminishes with age, so wounds are slower to heal. The thinning skin becomes vulnerable to injuries and damage. The underlying network of elastin and collagen fibers, which provides scaffolding for the surface skin layers, loosens and unravels. Skin then loses its elasticity. When pressed, it no longer springs back to its initial position but instead sags and forms furrows. The skin is more fragile and may bruise or tear easily and take longer to heal.

Mesenchymal stem cells (MSC) have potential to differentiate to lineages of mesenchymal tissues including bone, cartilage, fat, tendon, muscle, and marrow stroma. A variety of bone and cartilage disorders are known, and may be regenerated by mesenchymal stem cells. Included in such conditions is osteoarthritis. Osteoarthritis occurs in the joints of the body as an expression of “wear-and-tear”. Thus athletes or overweight individuals develop osteoarthritis in large joints (knees, shoulders, hips) due to loss or damage of cartilage. This hard, smooth cushion that covers the bony joint surfaces is composed primarily of collagen, the structural protein in the body, which forms a mesh to give support and flexibility to the joint. When cartilage is damaged and lost, the bone surfaces undergo abnormal changes. There is some inflammation, but not as much as is seen with other types of arthritis. Nevertheless, osteoarthritis is responsible for considerable pain and disability in older persons.

The term “muscle cell” as used herein refers to any cell which contributes to muscle tissue. Myoblasts, satellite cells, myotubes, and myofibril tissues are all included in the term “muscle cells”. Muscle cell effects may be induced within skeletal, cardiac and smooth muscles. Muscle tissue in adult vertebrates will regenerate from reserve myoblasts called “satellite cells”, or mesangioblasts, bone marrow derived cells, muscle interstitial cells, mesenchymal stem cells, etc. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers. One example of muscle stem cells is cells characterized as CD45⁻, CD11b⁻, CD31⁻, Scat, α7 integrin⁺, and CD34⁺.

In addition to skeletal muscle formation, the regeneration of cardiac muscle in the aging is of interest. For example, following an event such as myocardial infarction; surgery, catheter insertion, atherosclerosis, and the like, cardiac muscle can be damaged.

Ex vivo and in vitro differentiated cell populations useful as a source of cells may be obtained from any mammalian species, e.g. human, primate, equine, bovine, porcine, canine, feline, etc., particularly human cells. Ex vivo and in vitro differentiated cell populations may include fresh or frozen cells, which may be from a neonate, a juvenile or an adult, and differentiated tissues including skin, muscle, blood, liver, pancreas, lung, intestine, stomach, and other differentiated tissues. Pluripotent cells are optionally deleted from the differentiated cell population prior to introduction into the recipient. The dose of cells will be determined based on the specific nature of the cell, recipient and nature of condition to be treated, and will generally include from about 10⁶-10¹⁰ cells/kg body weight of the recipient, e.g. at least about 10⁶ cells/kg body weight; at least about 10⁷ cells/kg body weight; at least about 10⁸ cells/kg body weight; at least about 10⁹ cells/kg body weight; at least about 10¹⁰ cells/kg body weight; which may be provided in suspension, as aggregates, and the like.

To determine the suitability of cell compositions for therapeutic administration, the cells can first be tested in a suitable animal model. At one level, cells are assessed for their ability to survive and maintain their phenotype in vivo. Cell compositions may be administered to immunodeficient animals (such as nude mice, or animals rendered immunodeficient chemically or by irradiation). Tissues are harvested after a period of regrowth, and assessed as to whether the administered cells or progeny thereof are still present. This can be performed by administering cells that express a detectable label (such as green fluorescent protein, or β-galactosidase); that have been prelabeled (for example, with BrdU or [³H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

The term “cell culture” or “culture” means the maintenance of cells in an artificial, in vitro environment. Culture conditions may include, without limitation, a specifically dimensioned container, e.g. flask, roller bottle, plate, 96 well plate, etc.; culture medium comprising suitable factors and nutrients for growth of the desired cell type; and a substrate on the surface of the container or on particles suspended in the culture medium. By “container” is meant a glass, plastic, or metal vessel that can provide an aseptic environment for culturing cells.

The terms “primary culture” and “primary cells” refer to cells derived from intact or dissociated tissues or organ fragments. A culture is considered primary until it is passaged (or subcultured) after which it is termed a “cell line” or a “cell strain.” The term “cell line” does not imply homogeneity or the degree to which a culture has been characterized. A cell line is termed “clonal cell line” or “clone” if it is derived from a single cell in a population of cultured cells. Primary cells can be obtained directly from a human or animal adult or fetal tissue, including blood. The primary cells may comprise a primary cell line, or such as, but not limited to, a population of muscle satellite cells.

The terms “grafting”, “engrafting”, and “transplanting” and “graft” and “transplantation” as used herein refer to the process by which stem cells or other cells according to the present disclosure are delivered to the site where the cells are intended to exhibit a favorable effect, such as repairing damage to a patient's central nervous system, treating autoimmune diseases, treating diabetes, treating neurodegenerative diseases, or treating the effects of nerve, muscle and/or other damage caused by birth defects, stroke, cardiovascular disease, a heart attack or physical injury or trauma or genetic damage or environmental insult to the body, caused by, for example, disease, an accident or other activity. The stem cells or other cells for use in the methods of the present disclosure can also be delivered in a remote area of the body by any mode of administration as described above, relying on delivery of a magnetic force. For example, the term “cell engraftment” as used herein can refer to the process by which cells such as, but not limited to, muscle stem cells, are delivered to, and become incorporated into, a differentiated tissue such as a muscle, and become part of that tissue. For example, muscle stem cells, when delivered to a muscle tissue, may proliferate as stem cells, and/or may bind to skeletal muscle tissue, differentiate into functional myoblasts cells, and subsequently develop into functioning myofibers.

Lipid Structure.

One or more magnetic nanoparticles are encapsulated in a lipid, usually liposomal structure. Lipid structures can be important for maintaining the activity of lipophilic agents, can comprise growth factors or other agents, and may protect stem cell viability following in vivo administration. A liposome is a spherical vesicle with a membrane composed of a phospholipid bilayer. Liposomes can be composed of naturally-derived phospholipids with mixed lipid chains (like egg phosphatidylethanolamine), or of pure surfactant components like DOPE (dioleolylphosphatidylethanolamine). Liposomes often contain a core of encapsulated aqueous solution, and the magnetic microparticlex. The lipids may be any useful combination of known liposome or micelle forming lipids, including cationic lipids, such as phosphatidylcholine, or neutral lipids, such as cholesterol, phosphatidyl serine, phosphatidyl glycerol, and the like.

Suitable lipids include fatty acids, neutral fats such as triacylglycerols, fatty acid esters and soaps, long chain (fatty) alcohols and waxes, sphingoids and other long chain bases, glycolipids, sphingolipids, carotenes, polyprenols, sterols, and the like, as well as terpenes and isoprenoids. For example, molecules such as diacetylene phospholipids may find use. Included are cationic molecules, including lipids, synthetic lipids and lipid analogs, having hydrophobic and hydrophilic moieties, a net positive charge, and which by itself can form spontaneously into bilayer vesicles or micelles in water. Lipids may include, for example DSPC, DSPE, cholesterol, etc. Liposomes manufactured with a neutral charge, e.g. DMPC, can be used. Any amphipathic molecules that can be stably incorporated into lipid micelle or bilayers in combination with phospholipids can be used, with its hydrophobic moiety in contact with the interior, hydrophobic region of the micelle or bilayer membrane, and its polar head group moiety oriented toward the exterior, polar surface of the membrane.

The term “cationic amphipathic molecules” is intended to encompass molecules that are positively charged at physiological pH, and more particularly, constitutively positively charged molecules, comprising, for example, a quaternary ammonium salt moiety. Cationic amphipathic molecules typically consist of a hydrophilic polar head group and lipophilic aliphatic chains. Similarly, cholesterol derivatives having a cationic polar head group may also be useful. See, for example, Farhood et al. (1992) Biochim. Biophys. Acta 1111:239-246; Vigneron et al. (1996) Proc. Natl. Acad. Sci. (USA) 93:9682-9686. Cationic amphipathic molecules of interest include, for example, imidazolinium derivatives (WO 95/14380), guanidine derivatives (WO 95/14381), phosphatidyl choline derivatives (WO 95/35301), and piperazine derivatives (WO 95/14651). Examples of cationic lipids that may be used in the present invention include DOTIM (also called BODAI) (Saladin et al., (1995) Biochem. 34: 13537-13544), DDAB (Rose et al., (1991) BioTechniques 10(4):520-525), DOTMA (U.S. Pat. No. 5,550,289), DOTAP (Eibl and Wooley (1979) Biophys. Chern. 10:261-271), DMRIE (Feigner et al., (1994) J. Bioi. Chern. 269(4): 2550-2561), EDMPC (commercially available from Avanti Polar Lipids, Alabaster, Ala.), DCC hoi (Gau and Huang (1991) Biochem. Biophys. Res. Comm. 179:280-285), DOGS (Behr et al., (1989) Proc. Natl. Acad. Sci. USA, 86:6982-6986), MBOP (also called MeBOP) (WO 95/14651), and those described in WO 97/00241.

In some embodiments, the vesicle-forming lipid is selected to achieve a specified degree of fluidity or rigidity, to control the stability of the structure in serum, etc. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. Lipid fluidity is achieved by incorporation of a relatively fluid lipid, typically one having a lipid phase with a relatively low liquid to liquid-crystalline phase transition temperature, e.g., at or below room temperature.

The liposomes may be prepared by a variety of techniques, such as those detailed in Szoka, F., Jr., et al., Ann. Rev. Biophys. Bioeng. 9:467 (1980). Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium containing the desired magnetic nanoparticles for encapsulation. The lipid film hydrates to form MLVs, e.g., in some cases with sizes in a range of from 0.1 to 10 microns.

The liposomes, micelles, etc. of the disclosure may have substantially homogeneous sizes in a selected size range, for example, between 0.005 to 0.5 microns (e.g., 0.01 to 0.5 0.02 to 0.5, 0.025 to 0.5, 0.05 to 0.5, 0.075 to 0.5, 0.1 to 0.5, 0.005 to 0.4, 0.01 to 0.4 0.02 to 0.4, 0.025 to 0.4, 0.05 to 0.4, 0.075 to 0.4, 0.1 to 0.4, 0.005 to 0.3, 0.01 to 0.3 0.02 to 0.3, 0.025 to 0.3, 0.05 to 0.3, 0.075 to 0.3, 0.1 to 0.3, 0.005 to 0.2, 0.01 to 0.2 0.02 to 0.2, 0.025 to 0.2, 0.05 to 0.2, 0.075 to 0.2, 0.1 to 0.2, 0.005 to 0.1, 0.01 to 0.1 0.02 to 0.1, 0.025 to 0.1, 0.05 to 0.1, 0.075 to 0.1, 0.02 to 0.05, or 0.02 to 0.35 microns). In some embodiments vesicles have an average size of from about 50 nm, about 100 nm, about 150 nm up to about 750 nm, up to about 500 nm, up to about 400 nm.

One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less.

The number of magnetic nanoparticles encapsulated in a liposome will vary depending on the size of the liposome, and the concentration of magnetic particles in the aqueous medium.

The pharmaceutical compositions of the present disclosure can also comprise a pharmaceutically acceptable carrier. Many pharmaceutically acceptable carriers may be employed in the compositions of the present disclosure. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The concentration of liposomes in the carrier may vary. Generally, the concentration can be about 0.1 to 1000 mg/ml, usually about 1-500 mg/ml, about 5 to 100 mg/ml, etc. Persons of skill may vary these concentrations to optimize treatment with different lipid components or of particular patients.

The methods used for tethering stem cells to the external surface of a liposome or micelle can utilize a covalent attachment moiety or a non-covalent attachment moiety, e.g. a biotin/avidin or streptavidin pair. In some cases, crude liposomes are first pre-formed and a lipophilic agent, followed by various formulation steps, which may include size filtering; dialysis, and the like.

Magnetic Nanoparticles.

Superparamagnetic iron oxide nanoparticles are typically composed of a magnetite (Fe₃O₄) or maghemite (γ-Fe₂O₃) core. Both are naturally ferromagnetic in bulk, meaning they are permanently attracted to magnets or are permanently magnetic, but at diameters smaller than their intrinsic superparamagnetic radius and greater than their single domain radius, they become superparamagnets. Classification of these superparamagnets depends on the field of study as well as their application. In the field of medicine and biology, they are categorized broadly by hydrodynamic size; (50-180 nm) superparamagnetic iron oxide NPs (SPIONs), (10-50 nm) ultra-small superparamagnetic iron oxide NPs (USPIONs), and (<10 nm) very small superparamagnetic iron oxide NPs (VSPIONs).

SPIONs are sensitive in the nanomolar range and can be detected by T1, T2, and T2* MRI parameters vividly. SPIONs have negligible side effects when administered in vivo. Iron containing nanoparticles show an acceptable level of biocompatibility in part due to the body's innate ability to metabolize naturally occurring iron in the form of ferritin, and because they are superparamagnetic, the risk of particle agglomeration and thus vessel occlusion, is minimized. Further biocompatibility can be achieved by coating the cores with both inorganic and organic polymers. FDA approved and clinically viable SPIONs in the US market include Gastromark (50 nm), also known as Lumirem; and Ferumoxytol (20-50 nm), see Mahmoudi et al. (2011) Adv. Drug Deliv. Rev. 63:24-46.

There are a number of ways in which magnetic nanoparticles can be synthesized. Each offers discerning benefits in terms of quality, size distribution, and stability of the particles formed. The synthesis of iron oxides using co-precipitation works by reacting aqueous salt solutions of either Fe²⁺ or Fe³⁺ with a base at room temperature in an inert atmosphere to form magnetite (Fe₃O₄), which is then easily oxidized into maghemite γ-Fe₂O₃, its stable counterpart. Nanoparticle properties, including shape, size, and composition depend on the type of salt used in the reaction. Thus, iron chlorides, sulfates, and nitrates will confer different qualities onto the iron oxide particles formed. The co-precipitation process is relatively simple, which makes it among the most popular methods for producing iron oxide nanoparticles. Its main drawback is its inability to produce a narrow particle size distribution. While it is possible to control the size of the particle by altering stirring speeds and synthesis temperatures during production, the size distribution will vary within the range of one order of magnitude.

Superparamagnetism depends on blocking temperature, which is a factor of particles size, as well as the effective anisotropy constant, the applied magnetic field, and the experimental measuring time. Above this temperature, the thermal energy of the particles is high enough to randomize their magnetic moments, leading to a superparamagnetic state; below this temperature, they behave like permanent magnets. Body temperature and the Boltzmann constant are also fixed quantities, thus the size of the particles and the magnetic field applied on them will be the most relevant variables in determining a nanoparticle's superparamagnetism.

Magnetic nanoparticles have an innate instability in air causes them to oxidize, a process that alters the size of the particle. Various coating may be used including, for example, mild oxidation, surfactants, precious metals, silica, carbon coatings, cellulose, chitosan, etc. In some embodiments the coating is selected from polyethylene glycol (PEG), starch, citrate and dextran.

Magnetic nanoparticles usable in this invention can use, as a main component, any one of magnetite, Fe₂O₃, Fe₃O₄, mixed ferrite, and other iron-containing compounds including organic ferromagnetic material. Of these, ferrite, Fe₃O₄ exhibiting a maximum force, which is superior in magnetic responsibility, is specifically preferred. There was developed a technique in which nano-sized nanoparticles of ferrite (solid solution of Fe₃O₄ and δ-Fe₂O₃) exhibiting superior magnetic characteristics were synthesized by a controlled precipitation method under mild conditions of a temperature of 4 to 25° C. and a neutral pH. The preparation of this invention employs such mixed ferrite nanoparticles as suitable magnetic nanoparticles. Magnetic nanoparticles having the foregoing ferrite as a core can further contain various metal elements such as Zn, Co and Ni to control magnetic characteristics. The average particle size of the magnetic nanoparticles is usually from 1 to 30 nm, preferably from 5 to 25 nm, and more preferably from 5 to 20 nm. The number of nanoparticles in a vesicle may range, for example at least about 5, at least about 10, at least about 15 and up to about 100, up to about 75, up to about 50, up to about 30.

### Compositions and Methods

A composition comprising magnetic vesicular particles is provided, where magnetic nanoparticles are encapsulated by a liposome, which liposome comprises a binding moiety suitable for attachment to a stem cell of interest. The binding moiety may be covalent, e.g. an EDC linker, thiol linkage, etc. or a lipid of the liposome may be modified to comprise a binding moiety, e.g. biotin, a binding peptide, etc., binding moiety such an antibody or fragment thereof specific for CD34, and the like.

Lipid head groups useful to bind to targeting moieties include, for example, biotin, amines, cyano, carboxylic acids, isothiocyanates, thiols, disulfides, ahalocarbonyl compounds, unsaturated carbonyl compounds, alkyl hydrazines, etc. Chemical groups that find use in linking a targeting moiety to an stem cell surface molecule include carbamate such as EDC; amide (amine plus carboxylic acid); ester (alcohol plus carboxylic acid), thioether (haloalkane plus sulfhydryl; maleimide plus sulfhydryl), Schiffs base (amine plus aldehyde), urea (amine plus isocyanate), thiourea (amine plus isothiocyanate), sulfonamide (amine plus sulfonyl chloride), disulfide; hyrodrazone, lipids, and the like, as known in the art. For example, targeting may be achieved by converting a commercially available lipid, such as DAGPE, a PEG-PDA amine, DOTAP, etc. into an isocyanate, followed by treatment with triethylene glycol diamine spacer to produce the amine terminated thiocarbamate lipid which by treatment with the para-isothiocyanophenyl glycoside of the targeting moiety produces the desired targeting glycolipids. This synthesis provides a water soluble flexible linker molecule spaced between the lipid molecule that is integrated into the nanoparticle, and the ligand that binds to cell surface receptors, allowing the ligand to be readily accessible to the protein receptors on the cell surfaces.

Compositions are also provided of a regenerative cell population, e.g. a stem cell, progenitor cell, etc. bound to a magnetic vesicular particle through a binding moiety. The magnetic vesicular particles contain at least one magnetic microparticle as a ferrite core. The number of magnetic nanoparticles is variable depending on the average size of magnetic nanoparticles, the average size of magnetic vesicular particles and magnetic characteristics required as the preparation of this invention, therefore, the number of magnetic, nanoparticles is optimally adjusted. The average size of magnetic vesicular particles is usually from 50 to 300 nm, preferably from 50 to 200 nm, and from 50 to 150 nm.

An effective dose of a regenerative cell population may be formulated to delivery to a subject in need thereof. The differentiated, progenitor or stem cells may be used for tissue reconstitution or regeneration in a human patient or other subject in need of such treatment. The cells are administered in a manner that permits them to circulate, and to be localized through application of a magnet to the intended tissue site and reconstitute or regenerate the functionally deficient area.

A feature of the invention is the ability to localize stem cells through application of a magnetic field at the targeted tissue site. For example, focused cellular migration into a pocket of neutral magnetism can be achieved by aiming two NdFeB magnets, with alike poles facing each other, in the direction of the target site. A neodymium boron permanent disk magnet with a permanent magnetic actuator design that increased the force of the magnetic field at distances farther away from the point of actuation has been used for this purpose. A gold-plated neodymium boron magnetic disk can be used.

The differentiated, progenitor or stem cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or feeder cells associated with progenitor cell proliferation and differentiation.

The cells of this invention can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells.

Cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured.

The cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type.

Some specific regenerative methods may include, for example, delivery of mesenchymal or neural stem cells for restorative therapy for spinal cord injury in humans. The cells may be delivered, for example, by intrathecal injection. Stem or progenitor cells can be delivered to the site of vascular injury using external magnetic devices to help regenerate damage to endothelial layers, e.g. endothelial progenitor cells, mesenchymal stem cells, cardiac muscle stem cells, etc. Retinopathies can be treated by delivery and localization of stem cells to a dystrophic area of the retina without the use of invasive intraocular surgery, for example by magnetic targeting at the upper retinal hemisphere. Cartilage regeneration, e.g. introduction of mesenchymal stem cells, chondrocytes and cartilaginous stem or progenitor cells can be introduced into regions of cartilage defects, e.g. by intra-articular injection.

Following introduction of the magnetically labeled stem cells, a magnetic field at the desired tissue may be applied. The field may be maintained for a period of time sufficient to localize the cells, e.g. for at least about 1 hour, at least about 2 hours, at least about 4 hours, at least about 6 hours, at least about 12 hours, at least about 1 day, at least about 2 days, at least about 3 days, or more.

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody or cocktail of antibodies. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

The present invention will be further described with reference to specific examples but the invention is by no means limited to these.

### Example 1

### Liposome Loaded with Magnetic Nanoparticles for Targeted Delivery of Stem Cells to the Skin, Heart, Brain and Other Tissues

The ability to deliver stem cells to a target tissue, enhance stem cell signaling, and limit stem cell delocalization are important aspects in improving stem cell related products. Herein is provided a hybrid nanovesicle containing magnetic nanoparticles, which addresses these issues in stem cell delivery.

Superparamagnetic Iron Oxide Nanoparticles (SPIONs) were loaded within the liposome nanovesicles. The cells complexed with the nanovesicles (are then injected intravenously, and targeted to the desired tissue using MRI, strong NdFeB magnet, or Transcranial Magnetic Stimulation (TMS).

The hybrid nanovesicle structure has multiple benefits: The presence of SPIONs provide targeting capability for the stem cells, and also provides tracking through Magnetic Resonance Imaging (MRI) (Liao, N. et al. 2016 Sci. Rep. 6:18746). The liposome structure provides a means of including growth factors and therapeutic agents, and also provides a protecting layer that enhances SCs viability in vivo.

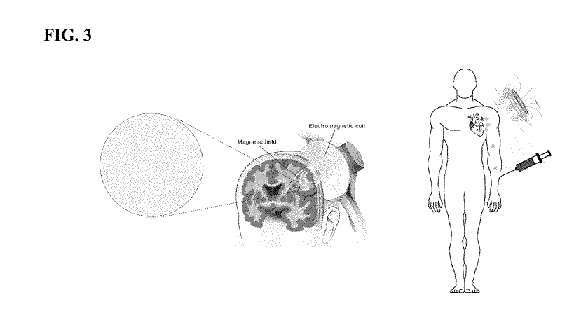

Targeted tissues may include the heart and brain using MRI, strong NdFeB magnet, or TMS (FIG. 3). Due to the superparamagnetic behavior of the nanoparticles encapsulated in the liposomes, controlled release of therapeutic agents within the liposome volume may be achieved using magnetic agitation of nanoparticles to generate heat, usually known as hyperthermia.

### Materials and Methods

Synthesis of Dextran Coated SPION.

Iron oxide nanoparticles were synthesized via co-precipitation process based on a previous report with some modification. 1 mmol (0.198 g) FeCl₂.4H₂O and 2 mmol (0.540 g) FeCl₃.6H₂O were added into a reactor containing 0.2 g dextran dissolved in 100 ml deionized H₂O, previously deoxygenized by Nitrogen bubbling for 20 min. They were stirred for 30 minutes under the Nitrogen bubbling by means of a magnetic stirring to ensure the proper dissolving of all agents. Gradually, salt solution temperature was risen to 80° C. using hot-plate apparatus. Then, 2.5 mM (0.1 g/mL) NaOH solution in DI H₂O (deoxygenized via Nitrogen gas for 5 mins) was quickly dropped into the solution and the hot-plate was removed. Black precipitates of dark suspension were dialyzed against PBS 1× overnight for two times.

Preparation of Magnetic Liposome.

10 mg of 1,2-Distearoyl-sn-glycero-3-phosphocholine (DSPC), 5 mg of cholesterol, and 2 mg 1,2-Distearoyl-sn-glycero-3-phosphoethanolamine (DSPE)-PEG-NH₂ were dissolved in 1 mL of chloroform. The transparent solution was poured into a 25 mL round bottom flask. Chloroform was evaporated using a vacuum rotary pump. 0.5 mL of dextran coated SPIONs added to the flask in an ultrasonic bath, while rotating. The temperature of bath was kept low with ice.

Linking Stem Cells to Magnetic Liposomes.

Thiol-maleimide chemistry was used to covalently bind stem cells to magnetic liposomes. This chemistry has a high reaction kinetic in biologically-compatible media, such as water and biological buffers. First, PBS-EDTA coupling buffer was prepared (50 mM Phosphate, 0.15 M NaCl, 10 mM EDTA, pH 7.2). 2 mg Crosslinker (Sulfo-SMCC) was added to 1 ml of PBS-EDTA. The solution was used immediately to avoid hydrolysis. Then, liposomes (1 mM) were added to the solution and mixed for 1 h at room temperature. Next, 100 μL of HEK 293 cells (1.25×104 cells/mL) were mixed with 1 mL of the solution for 2 h. Finally, cells were retrieved via 5 min centrifugation and resuspended in cell culture.

The ability to crosslink primary amines to carboxylic acid groups by EDC is a widespread tool for crosslinking peptides and proteins, and biological moieties. Components of the liposome contain primary amine group, and cells have proteins containing carboxylic acids. Therefore, EDC chemistry is applicable to link liposomes to stem cells. In addition to this chemistry, and in the quest of milder chemistry, conjugating via anti-CD34, or maleimide-thiol chemistry are also available for linking stem cells with our magnetic liposomes.

Targeted Delivery of SCs.

Once liposomes attach SCs properly, they are injected intravenously. Then, by using MRI, TMS, or a NdFeB magnet, SCs are delivered to any desired tissue.

What is claimed is:

1. A method of delivering a stem cell population to a targeted tissue, the method comprising:
administering to a subject in need thereof an effective dose of a stem cell population bound to a magnetic vesicle, comprising: one or more superparamagnetic nanoparticles encapsulated in the aqueous phase within a liposome; and
localizing the stem cell population to a targeted tissue by application of a magnetic field.

2. The method of claim 1, wherein the magnetic vesicle is bound to the stem cell population by a covalent linkage.

3. The method of claim 1, wherein the magnetic vesicle is bound to the stem cell population by a non-covalent linkage.

4. The method of claim 3, wherein the magnetic vesicle comprises biotin moieties, and the stem cell population comprises an avidin or streptavidin moiety.

5. The method of claim 1, wherein the superparamagnetic nanoparticles are comprised of ferrite.

6. The method of claim 1 wherein the superparamagnetic nanoparticles have an average particle size of from about 5 to about 20 nm in diameter.

7. The method of claim 1, wherein the magnetic vesicles have an average particle size of from about 100 to about 500 nm in diameter.

8. The method of claim 1, wherein the magnetic vesicles comprise from about 5 to about 50 nanoparticles.

9. The method of claim 1, wherein the magnetic field is generated by MRI, NdFeB magnet, or transcranial magnetic stimulation, where they can generate magnetic fields larger than 10 kOe.
